(19) United States
(12) Patent Application Publication
(10) Pub. No.: US 2023/0142073 A1
(43) Pub. Date: May 11, 2023

(54) Almitrine for treating the hypoxemia of a coronavirus infection

(71) Applicant: Michael David Forrest, Poole (GB)

(72) Inventor: Michael David Forrest, Poole (GB)

(21) Appl. No.: 17/911,823

(22) PCT Filed: Mar. 15, 2021
(86) PCT No.: PCT/IB2021/052138

§ 371 Date: Sep. 15, 2022

(30) Foreign Application Priority Data
Mar. 15, 2020 (GB) 2003731.3
Mar. 15, 2020 (GB) 2003732.1
Apr. 1, 2020 (GB) 2004778.3

(51) Int. Cl. A61K 31/53; A61K 33/00; A61K 31/573; A61K 31/675; A61P 35/00; A61P 11/00; A61P 31/14; A61P 9/10; A61K 45/06

(52) U.S. Cl. CPC A61K 31/53; A61K 33/00; A61K 31/573; A61K 31/675; A61P 35/00; A61P 11/00; A61P 31/14; A61P 9/10; A61K 45/06

(57) ABSTRACT

Hypoxemia is an abnormally low O₂ concentration in the blood. It can be lethal. When vital organs don't get enough O₂ to operate properly. A coronavirus infection can cause hypoxemia. Wherein this is probably the most common cause of coronavirus associated death. COVID-19 has killed millions of people to date. A method comprising the administration of a therapeutically effective amount of almitrine dimesylate to a subject with a coronavirus infection. To treat/ameliorate/reduce/prevent any coronavirus associated hypoxemia in the subject. Therein increasing the subject's chance of survival.

### BACKGROUND

A coronavirus infection can cause respiratory problems in a subject. For example, SARS-CoV-2, the coronavirus responsible for the COVID-19 pandemic, can cause breathing disorder/difficulty (and/or low PaO₂) in some subjects, which can be lethal in a fraction of cases. Killing millions so far.

### SUMMARY

A method comprising the administration of a therapeutically effective amount of almitrine, and/or a pharmaceutically-acceptable salt thereof, e.g. almitrine dimesylate, to a subject with (or suspected to have, or at risk of) a coronavirus infection, for the treatment/amelioration/prevention of at least one respiratory symptom of a coronavirus infection (e.g. one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation) and/or to reduce the probability of coronavirus killing the subject (increasing the subject's chance of survival). In some embodiments, the subject is human, and the coronavirus is SARS-CoV-2.

PaO₂ is the partial pressure of O₂ in arterial blood. A coronavirus (e.g. SARS-CoV-2) infection can reduce PaO₂ in a subject. Reduced PaO₂ is called hypoxemia (the term can also encompass low pO₂ in the blood more generally), and severely reduced PaO₂ is called anoxemia. Hypoxemia/anoxemia is probably the biggest danger in a coronavirus infection. Because not enough O₂ is delivered to the tissues (tissue hypoxia/anoxia). Which is probably the greatest cause of coronavirus associated deaths. By the teaching of this present disclosure, administering a therapeutically effective amount of almitrine, and/or a pharmaceutically-acceptable salt thereof, e.g. almitrine dimesylate, to a coronavirus (e.g. SARS-CoV-2) infected subject treats/ameliorates/reduces/prevents any pathological reduction of their PaO₂ value (any hypoxemia/anoxemia) caused by their coronavirus infection.

### DETAILED DESCRIPTION OF THE INVENTION

A method of treating/ameliorating/preventing at least one respiratory symptom (non-limiting e.g. one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation) of a coronavirus infection in a subject (non-limiting e.g. a human subject) comprising administering to the subject (and/or the subject self-administering) a therapeutically effective amount of at least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I).

Use of at least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I), in the manufacture of a medicament for treating/ameliorating/preventing at least one respiratory symptom (non-limiting e.g. one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation) of a coronavirus infection in a subject (e.g. a human subject). Optionally wherein the medicament is in a ready-to-use drug form, optionally in a package/packaging together with instructions for its use in a subject with a coronavirus (e.g. SARS-CoV-2) infection (optionally that has hypoxemia), optionally distributed as a kit for oral and/or intravenous administration.

At least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I), for use in a method of treating/ameliorating/preventing at least one respiratory symptom (non-limiting e.g. one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation) of a coronavirus infection in a subject (e.g. a human subject).

Formula (I) is presented later in this disclosure, under the heading of “Formula (I)”.

A method comprising the administration of a therapeutically effective amount of at least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I), to a subject with (or suspected to have, or at risk of) a coronavirus (e.g. SARS-CoV-2) infection (or other viral infection that can cause one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation, in an infected subject).

A method of reducing the probability of a subject's (who is optionally on mechanical ventilation [invasive or non-invasive], optionally on elevated O₂ in their breathing gas) coronavirus (e.g. SARS-CoV-2) infection killing them (increasing the subject's chance of survival) and/or reducing their probability of requiring supplemental oxygen (oxygen therapy) and/or reducing their probability of requiring Mechanical Ventilation (MV) and/or reducing their probability of requiring Extracorporeal Membrane Oxygenation (ECMO) and/or reducing their probability of requiring respiratory support and/or reducing their probability of requiring hospitalization/Intensive Care Unit (ICU) and/or reducing their time required upon/in one or more of supplemental oxygen, MV, ECMO, respiratory support, hospital, ICU and/or delaying their need for one or more of supplemental oxygen, MV, ECMO, respiratory support, hospitalization, ICU and/or reducing their time to recovery (e.g. discharged from hospital sooner) and/or reducing the duration that they require respiratory support (if applicable) and/or (if they are on respiratory support) augmenting their respiratory support (non-limiting e.g. augmenting one or more of artificial/mechanical ventilation [invasive or non-invasive], supplemental oxygen (oxygen therapy), Extracorporeal Membrane Oxygenation etc.) and/or improving their clinical outcome comprising administering to the subject (and/or the subject self-administering) a therapeutically effective amount of at least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I).

Use of at least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I), in the manufacture of a medicament for reducing the probability of a subject's (who is optionally on mechanical ventilation [invasive or non-invasive], optionally on elevated O₂ in their breathing gas) coronavirus (e.g. SARS-CoV-2) infection killing them (increasing the subject's chance of survival) and/or reducing their probability of requiring supplemental oxygen (oxygen therapy) and/or reducing their probability of requiring Mechanical Ventilation (MV) and/or reducing their probability of requiring Extracorporeal Membrane Oxygenation (ECMO) and/or reducing their probability of requiring respiratory support and/or reducing their probability of requiring hospitalization/Intensive Care Unit (ICU) and/or reducing their time required upon/in one or more of supplemental oxygen, MV, ECMO, respiratory support, hospital, ICU and/or delaying their need for one or more of supplemental oxygen, MV, ECMO, respiratory support, hospitalization, ICU and/or reducing their time to recovery (e.g. discharged from hospital sooner) and/or reducing the duration that they require respiratory support (if applicable) and/or (if they are on respiratory support) augmenting their respiratory support (non-limiting e.g. augmenting one or more of artificial/mechanical ventilation [invasive or non-invasive], supplemental oxygen (oxygen therapy), Extracorporeal Membrane Oxygenation etc.) and/or improving their clinical outcome. Optionally wherein the medicament is in a ready-to-use drug form, optionally in a package/packaging together with instructions for its use in a subject with a coronavirus (e.g. SARS-CoV-2) infection (optionally that has hypoxemia), optionally distributed as a kit for oral and/or intravenous administration.

At least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I), for use in a method of reducing the probability of a subject's (who is optionally on mechanical ventilation [invasive or non-invasive], optionally on elevated O₂ in their breathing gas) coronavirus (e.g. SARS-CoV-2) infection killing them (increasing the subject's chance of survival) and/or reducing their probability of requiring supplemental oxygen (oxygen therapy) and/or reducing their probability of requiring Mechanical Ventilation (MV) and/or reducing their probability of requiring Extracorporeal Membrane Oxygenation (ECMO) and/or reducing their probability of requiring respiratory support and/or reducing their probability of requiring hospitalization/Intensive Care Unit (ICU) and/or reducing their time required upon/in one or more of supplemental oxygen, MV, ECMO, respiratory support, hospital, ICU and/or delaying their need for one or more of supplemental oxygen, MV, ECMO, respiratory support, hospitalization, ICU and/or reducing their time to recovery (e.g. discharged from hospital sooner) and/or reducing the duration that they require respiratory support (if applicable) and/or (if they are on respiratory support) augmenting their respiratory support (non-limiting e.g. augmenting one or more of artificial/mechanical ventilation [invasive or non-invasive], supplemental oxygen (oxygen therapy), Extracorporeal Membrane Oxygenation etc.) and/or improving their clinical outcome.

Almitrine, or almitrine dimesylate, for treating/ameliorating/preventing Coronavirus Virus Infectious Disease (COVID, e.g. COVID-19) in a subject.

In some embodiments, almitrine (and/or a pharmaceutically-acceptable salt, solvate, hydrate or prodrug thereof, e.g. almitrine dimesylate) administration to a number of subjects with a coronavirus infection reduces their median mortality (e.g. median 30 day mortality), and in alternative embodiments, it only reduces the median mortality (e.g. median 30 day mortality) of a sub-set: only those requiring respiratory support (e.g. mechanical ventilation e.g. the invasive sub-type thereof). In other embodiments, in addition or instead, it reduces the median of one or more of hospitalization duration, rate of intensive care unit (ICU) admission, use of mechanical ventilation, or some composite endpoint thereof. Reducing the duration that a subject with coronavirus spends in hospital reduces the spread of coronavirus within the hospital, and thence the broader population (reduces subject's infectivity, reduces the number of other individuals [e.g. people] that the subject infects with coronavirus; when this is done for a population of coronavirus infected subjects it reduces the Reproduction (R) number of their coronavirus transmission to uninfected individuals, which can slow the growth rate of a coronavirus outbreak/epidemic/pandemic).

Some non-limiting examples of a coronavirus are betacoronavirus, SARS-CoV, SARS-CoV-2 {cause of COVID-19 pandemic}, MERS-CoV, HCoV-NL63, HCoV-229E, HCoV-0C43, HCoV-HKU1 etc. Wherein any coronavirus that can infect humans is especially contemplated. For non-limiting example, a SARS-like bat coronavirus that has made the species jump into humans, either directly, or indirectly via one or more other species. In some embodiments, the coronavirus infection, causing the respiratory disorder(s), is SARS-CoV-2. Which is the coronavirus responsible for the COVID-19 pandemic. All variants of SARS-CoV-2 are contemplated. For non-limiting example, Lineage P.1. Also known as 20J/501Y.V3, or Variant of Concern 202101/02 (VOC-202101/02), or colloquially known, at present, as the Brazil(ian) variant. With COVID-19, the world has now observed the mass, global mortality that a coronavirus can cause. The teaching of this disclosure can reduce the mortality of future coronavirus epidemics/pandemics, as well as the present pandemic, which is still very much ongoing at the time of writing. Especially with the growing numbers of re-infections. It might be that SARS-CoV-2 is such a moving target, mutating around immunity (e.g. vaccine conferred), that it will be with us now, at some level, as an ongoing problem.

In some preferred embodiments, the used compound of Formula (I) is almitrine. Almitrine's CAS number is 27469-53-0. It has the following structure:

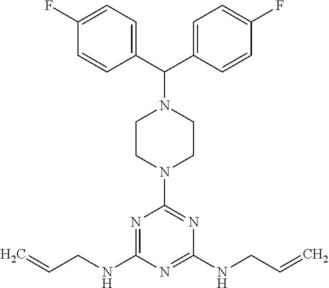 [SMILES: C=CCNc1nc(NCC=C)nc(N2CCN(C(c3ccc(F)cc3)c3ccc(F)cc3)CC2)n1]

And in further preferred embodiments, its dimesylate salt is used: almitrine dimesylate Almitrine dimesylate is 6-[4-[bis(4-fluorophenyl)methyl]piperazin-1-yl]-2-N,4-N-bis(prop-2-enyl)-1,3,5-triazine-2,4-diamine methanesulfonic acid, the dimethanesulfonate salt of almitrine. Almitrine dimesylate is also known as almitrine bismesylate or almitrine dimethanesulfonate. All pharmaceutical salts of almitrine are contemplated. As is almitrine in complex with another drug(s) e.g. almitrine-raubasine.

In some embodiments, almitrine, and/or other compound(s) of Formula (I), and/or a pharmaceutically-acceptable salt, solvate, hydrate or prodrug thereof (e.g. almitrine dimesylate), and/or a pharmaceutical composition(s) comprising one or compounds of Formula (I), is administered to a subject in co-therapy with one or more drugs and/or treatments licensed by one or more national/multinational regulatory authorities such as, for non-limiting example, the Food and Drug Administration (FDA) in the USA, European Medicines Agency (EMA) in Europe, National Medical Products Administration (NMPA) in China, Pharmaceuticals and Medical Devices Agency (PMDA) in Japan, Medicines and Healthcare products Regulatory Agency (MHRA) in the UK, or similar agency in another jurisdiction. Optionally wherein the other drug(s) and/or treatment(s) is licensed/used for one or more of anti-viral (non-limiting e.g. one or more of Remdesivir, GS-441524 (Gilead Sciences, Inc. drug), Lopinavir, Ritonavir, Molnupiravir, Kaletra, Thapsigargin, Favilavir, Glycyrrhizin, Favipiravir, β-D-N4-hydroxycytidine-5′-isopropyl ester, Remdesivir/baricitinib combination, Remdesivir/interferon beta-1a combination, lopinavir/ritonavir combination), anti-coronavirus, anti-respiratory disorder(s) (non-limiting e.g. is a respiratory stimulant drug {non-limiting e.g. doxapram, GAL021}), anti-inflammatory, immunosuppressant, anti-cytokine storm, androgen antagonist (non-limiting e.g. proxalutamide, bicalutamide), immunomodulator(s)/corticosteroid(s)/glucocorticoid(s) (non-limiting e.g. Dexamethasone and/or a pharmaceutically-acceptable salt, solvate, hydrate or prodrug thereof), anti-fever (e.g. paracetamol), peripheral neuropathy use/treatment, and/or is licensed for use in patients with a coronavirus infection e.g. with the licensed aim of improving their clinical outcome, and/or is an antibody drug thereof (non-limiting e.g. one or more of Bamlanivimab, Etesevimab, Sarilumab, Tocilizumab, anti-SARS-CoV-2 monoclonal antibod[y/ies]). Optionally wherein almitrine, and/or other compound(s) of Formula (I), and/or a pharmaceutically-acceptable salt, solvate, hydrate or prodrug thereof (e.g. almitrine dimesylate), is in the same pharmaceutical composition as one or more other drugs (e.g. one or more anti-inflammatory drugs [non-limiting e.g. Dexamethasone], and/or one or more anti-viral drugs [non-limiting e.g. Remdesivir]) administered in co-therapy. Wherein the therapeutic effect being greater than the sum of each individual drug alone (synergy) is contemplated and componentry to this disclosure. Particularly contemplated is co-therapy of a subject with a coronavirus (e.g. SARS-CoV-2) infection with almitrine (and/or a pharmaceutically-acceptable salt, solvate, hydrate or prodrug thereof, e.g. almitrine dimesylate) and dexamethasone (and/or a pharmaceutically-acceptable salt, solvate, hydrate or prodrug thereof) and/or remdesivir (and/or a pharmaceutically-acceptable salt, solvate, hydrate or prodrug thereof), optionally wherein they are administered in the same pharmaceutical composition (or, optionally, in separate pharmaceutical compositions but which are packaged/distributed/sold together) [optionally wherein these two or three drugs are co-administered with breathed NO {e.g. at 10 ppm}]. Wherein, for many coronavirus patients, those requiring respiratory support especially (e.g. mechanical ventilation e.g. the invasive sub-type thereof), this administered almitrine-dexamethasone (or almitrine-dexamethasone-remdesivir, or almitrine-remdesivir) combination can confer greater therapeutic effect for a subject's coronavirus infection than any of the drugs alone (e.g. greater reduction in 30 day mortality, e.g. faster recovery time [e.g. faster hospital discharge], e.g. shorter duration required upon respiratory support etc.). Wherein this being more than an additive effect, it being synergistic, is herein contemplated, and is componentry to this disclosure.

In subjects with a coronavirus infection, the almitrine-remdesivir combination can drive better clinical outcomes than remdesivir alone. An intravenous formulation containing both these drugs is contemplated herein.

In some embodiments, almitrine (and/or a pharmaceutically-acceptable salt, solvate, hydrate or prodrug thereof, e.g. almitrine dimesylate) is administered to a coronavirus infected subject whether they require respiratory support or not, and in other embodiments, only when they require respiratory support. In some embodiments, almitrine (and/or a pharmaceutically-acceptable salt, solvate, hydrate or prodrug thereof, e.g. almitrine dimesylate) is orally administered to a coronavirus infected subject when they don't need respiratory support, and intravenously when they do. In some embodiments, an almitrine containing composition, which also contains dexamethasone, is only administered to a coronavirus infected subject who requires respiratory support (illustrative e.g. invasive mechanical ventilation).

An embodiment(s) is administering almitrine and/or a pharmaceutically-acceptable salt, solvate, hydrate or prodrug thereof (e.g. almitrine dimesylate) to a subject (e.g. a human subject) with a coronavirus (e.g. SARS-CoV-2) infection that has difficulty breathing and/or has low arterial blood pO₂ (hypoxemia) and/or high arterial blood pCO₂. Optionally in co-administration with (e.g. inhalation/breathing of) Nitric Oxide, NO (illustratively, not restrictively, NO at 10 parts per million [p.p.m]), and/or hyperbaric O₂ therapy (oxygen therapy/supplemental oxygen, with or without ventilatory support) and/or mechanical/assisted ventilation (artificial invasive/non-invasive assistance to support breathing, optionally with intubation) and/or Extracorporeal Membrane Oxygenation (ECMO) and/or prone/postural/supine positioning.

A therapeutically effective amount of almitrine (and/or a pharmaceutically-acceptable salt, solvate, hydrate or prodrug thereof, e.g. almitrine dimesylate) can optionally be administered to a subject as a supplement to their oxygen and/or nitric oxide therapy and/or mechanical/assisted venitilation and/or extracorporeal membrane oxygenation, and/or as an assist to transitioning a subject off one or more of these, and/or as a substitute for one or more of these, and/or rendering a subject's hospitalization shorter (the subject's need for a hospital machine(s)/care is delayed and/or shortened) or unrequired. This is especially useful during an epidemic/pandemic when there might not be enough hospital machines/beds for everyone with need. Moreover, hospitals can be a hub of coronavirus spread. Disproportionally to those least fit to endure it, and to key healthcare workers also, which is a vicious combination. So keeping subjects with (or suspected to have, or at risk of, or particularly vulnerable to) coronavirus out of hospital as much as possible, either by delaying their admission, expediting their discharge or removing/mitigating their need for hospitalization, is valuable. Also, being a drug with such a low side-effect profile, especially when taken short-term, almitrine can be taken with atypically limited medical oversight if access to a doctor(s) becomes atypically limited Almitrine can be used to transition/wean subjects with coronavirus (e.g. SARS-CoV-2) infection off mechanical/assisted ventilation, freeing up ventilator machines quicker, heading off the need for a ventilator machine in more minor cases, and buying time to be allocated a ventilator machine in more serious cases. This can ease the most dangerous pinch point of a coronavirus epidemic/pandemic: not enough ventilator machines for those that need them. Almitrine (and/or a pharmaceutically-acceptable salt, solvate, hydrate or prodrug thereof, e.g. almitrine dimesylate) administration to a coronavirus infected patient can reduce their need for mechanical ventilation, in some cases such that they have no such need, which is very valuable because mechanical ventilation carries great dangers in and of itself. Potentially life threatening dangers. And even in the best case it tends to exert a physical and psychological toll upon the subject. Once a coronavirus infected patient requires mechanical ventilation, their chance of survival falls dramatically. Not just because of the coronavirus. But because there are dangers inherent to undergoing (particularly invasive) mechanical ventilation itself. Mitigating the need for mechanical ventilation increases the chance of surviving a coronavirus infection greatly. It reduces mortality (e.g. reduces 30 day mortality).

Also contemplated herein is prophylactic (e.g. post-exposure prophylactic) administration, with at least one compound of Formula (I) (e.g. almitrine), and/or a pharmaceutically-acceptable salt, solvate, hydrate or prodrug thereof (e.g. almitrine dimesylate), to/by a subject without a coronavirus infection, but at risk (e.g. higher risk because of an epidemic/pandemic) of a coronavirus infection. Especially if they have one or more risk factors (non-limiting e.g. one or more of advanced age, elderly, overweight, cancer, lung cancer, Non-Small Cell Lung Cancer [NSCLC], mesothelioma, emphysema, asthma, pre-existing respiratory disorder[s] etc.) predicting a poorer prognosis should they become infected with a coronavirus (e.g. SARS-CoV-2). Also contemplated is distribution of at least one compound of Formula (I) (e.g. almitrine), and/or a pharmaceutically-acceptable salt, solvate, hydrate or prodrug thereof (e.g. almitrine dimesylate), preferably in an oral form, to one or more subjects without a coronavirus infection, for them to keep close by/to hand (e.g. in their place of residence and/or work) so that they can start self-administering as soon as they think they have been infected with a coronavirus, and/or they start exhibiting one or more signs/symptoms of a coronavirus infection.

In some embodiments the subject is a human over the age of n, wherein n is a number (e.g. integer) over 40 and less than 200, optionally one of 40, 50, 60, 70, 80, 90, 100.

In some embodiments, a respiratory symptom of a coronavirus (e.g. SARS-CoV-2) infection in a subject, which is treated/ameliorated/prevented by their being administered with (and/or self-administering) at least one compound of Formula (I) (e.g. almitrine), and/or a pharmaceutically-acceptable salt, solvate, hydrate or prodrug thereof (e.g. almitrine dimesylate), is a PaO₂/FiO₂ ratio below n where n is a positive number below (different numbers in different embodiments) one or more of 100, 110, 120, 130, 140, 150, 160, 170, 180, 190, 200, 210, 220, 230, 240, 250, 260, 270, 280, 290, 300 mmHg (at 26.7 kPa).

Non-limiting embodiments of this disclosure include almitrine

dosages/formulations/compositions/salts/patterns of administration that have already been used in humans (for example, as reported in the literature). For non-limiting example, oral almitrine dimesylate, administered at a value selected between 50 to 400 mg per day. For non-limiting example, intravenous almitrine dimesylate at a flow rate selected from within the range 1-16 (or 1-25) μg/kg/min (optionally wherein an intravenous bolus injection precedes the steady infusion rate). Furthermore, higher or lower almitrine (e.g. almitrine dimesylate) doses for anti-coronavirus (and/or symptom[s] thereof) therapy, optionally administered orally, and/or intravenously, are further embodiments of this disclosure. Parallel oral and intravenous administration of almitrine dimesylate in the same subject is contemplated.

Supporting Clinical Data

Relating to almitrine and coronavirus, some supporting clinical studies, in supporting publications, which are all incorporated herein in their entirety by reference, wherein the earliest priority date of this present application (15 Mar. 2020) predates any of their publication dates:
[1] Losser M R et al. (22 May, 2020) Almitrine as a non ventilatory strategy to improve intrapulmonary shunt in COVID-19 patients. medRxiv.
[2] Losser M R et al. (4 Jun. 2020) Almitrine as a non-ventilatory strategy to improve intrapulmonary shunt in COVID-19 patients. Anaesthesia, Critical Care & Pain Medicine. 39(4):467-469.
[3] Cardinale M et al. (4 Jun. 2020) Effect of almitrine bismesylate and inhaled nitric oxide on oxygenation in COVID-19 acute respiratory distress syndrome. Anaesthesia, Critical Care & Pain Medicine. 39:471-2.
[4] Barthelemy R et al. (6 Jun. 2020) Efficacy of almitrine in the treatment of hypoxemia in Sars-Cov-2 acute respiratory distress syndrome. Chest. 158(5):2003-2006.
[5] Huette P et al. (2 Jul. 2020) Acute Cor pulmonale in COVID-19-Related ARDS: Improvement with almitrine infusion. Case Reports. 2(9):1311-1314.
[6] Bendjelid K et al. (9 Jul. 2020) Treating hypoxemic COVID-19 “ARDS” patients with almitrine: The earlier the better? Anaesthesia, critical care & pain medicine. 39(4):451-452.
[7] Caplan M et al. (22 Oct. 2020) Almitrine Infusion in Severe Acute Respiratory Syndrome Coronavirus 2-Induced Acute Respiratory Distress Syndrome: A Single-Center Observational Study. Critical Care Medicine. 49(2):e191-e198.
[8] Bagate F et al. (4 Nov. 2020) Rescue therapy with inhaled nitric oxide and almitrine in COVID-19 patients with severe acute respiratory distress syndrome. Annals of intensive care. 10(1):1-7.
[9] Payen D (13 Nov. 2020) Coronavirus Disease 2019 Acute Respiratory Failure Almitrine Drug Resuscitation or Resuscitating Patients by Almitrine? Critical Care Medicine. 49(2):387-389.
[10] Nair A S et al. (2021) Role of almitrine bismesylate in managing refractory hypoxemia in COVID19 acute respiratory distress syndrome. Saudi Journal of Anaesthesia. 15(1):76.

Physiologically, hypoxia in part of a lung (e.g. perhaps because of lung damage and/or fluid in that part) causes vasoconstriction in this lung part (“Hypoxic Pulmonary Vasoconstriction” [HPV]). So that more blood can flow instead to other lung parts that actually have appreciable O₂ to deliver to the blood. Pharmacologically, almitrine helps and increases this process. Thence increasing pO₂, and decreasing pCO₂, in the blood and tissues.

“The severity of hypoxemia in COVID-19 may be partly explained by the loss of HPV, which is a potent adaptation to hypoxemia in humans” [7].

With SARS-CoV-2 infection, the “dominant respiratory feature is profound and disproportionate hypoxemia” wherein the “main cause of this condition is a severe intrapulmonary oxygen shunt” [6], “indeed, it appears that the present SARS-CoV-2 pneumonitis induces a significant intrapulmonary capillary shunt resulting from a loss of hypoxic pulmonary vasoconstriction (HPV)” [6].

Hypoxemia can be defined as a PaO₂/FiO₂ (ratio between the oxygen level in the blood, PaO₂, and the oxygen concentration that is breathed, FiO₂ [fraction of inspired oxygen; molar or volumetric fraction of oxygen in the inhaled gas]) below 200 mmHg (at 26.7 kPa). Intravenous almitrine dimesylate (30-45 minutes of 4 μg/kg/min infusion rate followed by 30-45 minutes of 12 μg/kg/min infusion rate) increased PaO₂/FiO₂ in 8, out of 10, human patients (median age of 70 years old) with a coronavirus (SARS-CoV-2) infection, for which they were intubated and ventilated (before the study commenced), with 7 positioned in the supine, and 3 positioned in the prone [1, 2, 6]. Their median PaO₂/FiO₂ ratio increased from 135, before almitrine administration, to 215 mmHg afterwards: an increase of 80 mmHg Therein shifting the median value of these coronavirus infected subjects out of hypoxemia. With their cardiac index and right atrial pressures not affected. 7 control patients, which were positioned in the prone, intubated and ventilated, but not administered almitrine dimesylate, did not have an improved median PaO₂/FiO₂ ratio: “the absence of a spontaneous PaO₂ improvement over 8 hours in matched controls reinforced the credence in an almitrine effect” [2].

Intravenous almitrine dimesylate (2 μg/kg/min) was associated with an increased median PaO₂/FiO₂ (79 to 117 mmHg, p-value=0.001) in the following 6 hours, in 19 Sars-Cov-2 ARDS patients (79% of which were mechanically ventilated, with neuromuscular blockade) [4].

A 57-year-old woman with a SARS-CoV-2 infection presented with Severe Acute Respiratory Distress Syndrome (SARDS) [5]. Even after mechanical ventilation (with lung-protective settings), prone positioning, deep sedation, neuromuscular blockade, and inhaled nitric oxide at 10 ppm, her PaO₂/FiO₂ was 70 mmHg (PaO₂ of 63 mm Hg). Intravenous almitrine dimesylate (4 μg/kg/min, for 4 days) increased her blood oxygenation incredibly. One (1), two (2) and twelve (12) hours after almitrine infusion, her PaO₂/FiO₂ was 84, 216, and 283 mmHg respectively (her PaO₂ was 67, 130 and 170 mm Hg respectively). The woman survived. Despite having an incredibly low PaO₂/FiO₂ at one point. Wherein none of the other treatments tried were successful in raising her PaO₂/FiO₂ from this extremely dangerously low value. Only almitrine succeeded.

32 SARS-CoV-2 infected patients, exhibiting Acute Respiratory Distress Syndrome (ARDS) {94% were intubated}, received intravenous almitrine dimesylate (10 μg/kg/min, and if the patient responded to this, they were put on a maintenance dose, the mean of which was 1.65 μg/kg/min; in 75% of cases Nitric Oxide [NO] was inhaled during almitrine infusion; treatment was for 5 days for 10 of the responders, for 11 of the responders it was for less than 2 days). 66% of the patients were responders (PaO₂ increase of more than 20%) with a mean increase of 67% in PaO₂ [7, 9], with no significant side-effects, and 16% lower mortality. This is remarkable when it is considered that this almitrine intervention was only applied after other methods (ventilation, Positive End Expiratory Pressure [PEEP] titration, curarization, inhaled NO, and prone positioning) had failed to improve PaO₂. So, these were serious, advanced cases. Almitrine is likely to have greater benefit when administered earlier [6].

Nitric Oxide (NO)—almitrine combination (10 ppm NO inhaled, 10 μg/kg/min almitrine dimesylate intravenous infusion for 30 minutes) was associated with rapid and significant improvement of oxygenation in SARS-CoV-2 infected patients presenting with Severe Acute Respiratory Distress Syndrome (SARDS) [8]. This combination outperforms NO alone. Median PaO₂/FiO₂ was 102 mmHg at baseline, 124 mmHg after NO, and 180 mmHg after NO and almitrine (p<0.01). No ill effects observed. So, almitrine dimesylate increases PaO₂ in human patients with a coronavirus (SARS-CoV-2) infection [1, 2, 4, 5, 6, 7, 8, 9, 0].

SARS-CoV-2 infection decreases PaO₂, almitrine dimesylate increases PaO₂ in SARS-CoV-2 infected human patients [1, 2, 4, 5, 6, 7, 8, 9, 10].

Almitrine dimesylate treats/ameliorates/prevents the low PaO₂ caused by coronavirus (SARS-CoV-2) infection. Wherein low PaO₂ is very arguably the most dangerous symptom of coronavirus (SARS-CoV-2) infection. Probably responsible for most of (if not all) the many millions of lives it has taken (to date).

Demonstrably Inventive

This treatment effect of almitrine, for the respiratory disorder(s) of SARS-CoV-2 infection, was not recognized by a “Person having Ordinary Skill in the Art” (POSA) in the period after the filing date of my priority application, on 15 Mar. 2020, and before the papers cited above published. Even when POSA was told of it, and taught it. They dismissed it. What POSA thought at that time is known because, on 20 Mar. 2020, I applied (with a confidentiality agreement [CDA] in place prior) to the National Institute for Health Research (NIHR), a clinical research arm to the British National Health Service (NHS), to run a clinical trial, within the NHS, of almitrine in subjects with coronavirus associated respiratory disorder. To adjudicate whether to run such a trial, the NIHR sought the judgement of three (3) experts, which I individually, and collectively, term POSA herein. So, POSA in this case was three (3) independent verdicts from three (3) medical doctors. Who were each clinical research professors at leading research universities (and their teaching hospitals). One of which was the University of Oxford, which is consistently ranked as one of the very best universities in the world (e.g. ranked 1^{st} in the Times Higher Education World University Rankings 2020). Also, these 3 POSAs were also all respiratory medicine consultants within the British National Health Service (NHS). Where “consultant” is the most senior doctor grade within the NHS. It means the doctor is a top specialist in a speciality. Wherein in this case, the speciality of the 3 POSAs was respiratory medicine. They each had published >100 peer-reviewed papers within this area, garnering many citations. So, these 3 POSAs were each at the pinnacle of clinical research, and clinical practice, within respiratory medicine. Each selected, because of this expertise, by the National Institute for Health Research (NIHR) {independent confirmation of these 3 POSAs being experts}, to assess my almitrine teaching (of using it to help patients with coronavirus, who experience respiratory difficulties). Which they dismissed. Saying that almitrine, in this context, was “not advised for use”.

I received this verdict, via the NIHR, on 30 Mar. 2020. After the earliest priority date of this present application, which is 15 Mar. 2020. So, even after its earliest priority date, my invention was not recognized by POSA. Because it was inventive at that time. Indeed, even with the invention in hand, POSA couldn't recognize it. Three (3) esteemed, leading, physician-scientists, each with bleeding-edge clinical research expertise in the area of respiratory medicine.

Longstanding, Unmet Need

Prior to the COVID-19 pandemic (caused by SARS-COV-2), a number of coronavirus epidemics/outbreaks have happened within the last 20 years. Principally, the SARS epidemic (caused by SARS-COV-1) in 2002-2004. And MERS-COV outbreaks have happened a number of times since 2012, most recently in 2018. Wherein these other coronavirus infections can also cause respiratory disorder(s) in humans. And future coronaviruses, which jump from another species (non-limiting e.g. bat or palm civet species) into humans, are very likely to also. Almitrine is a drug known since the 1970s. Before this present disclosure, with its earliest priority date of 15 Mar. 2020, no one had invented the method of using almitrine to treat/ameliorate/prevent the respiratory disorder(s) of a coronavirus infection. Despite much research, and a widely-acknowledged need to improve clinical outcomes, in this area.

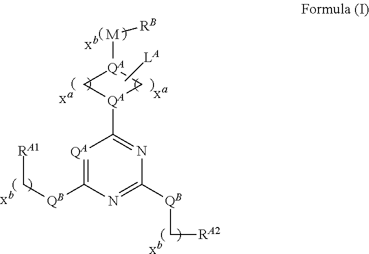

including

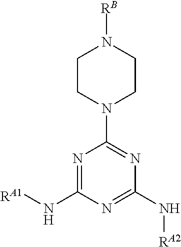

or a pharmaceutically-acceptable salt, solvate, hydrate or prodrug thereof, wherein:

each Q^{A} is independently selected from N and CH;

each Q^{B} is independently selected from O, S, Se, NH, CH₂, NR^{W}, PR^{W}, BR^{W}, C(R^{W})₂ and Si(R^{W})₂;

each M is independently selected from O, S, Se, NH, CH₂, NR^{W}, PR^{W}, BR^{W}, C(R^{W})₂ and Si(R^{W})₂;

each R^{W} is independently selected from hydrogen, deuterium, halogen (e.g. F), alkyl, or substituted alkyl (non-limiting examples: CF₃, CCl₃), or deuterated alkyl (non-limiting example: CD₃), or aminoalkyl, or thioalkyl, or alkoxy, or halogen, or haloalkyl, or haloalkoxy;

x^{a} is independently at each point of use selected from 1, 2, 3, 4, or 5; x^{b} is independently at each point of use selected from 0, 1, 2, 3, 4, or 5; L^{A} represents 0-5 optional substituents on the ring independently selected from alkyl, substituted alkyl, deuterated alkyl, aminoalkyl, thioalkyl, alkoxy, halogen, haloalkyl, haloalkoxy, or any atom or isotope permitted by valence (including any accompanying hydrogens by valence e.g. (non-limiting) OH, NH₂, SH, SiH₃, PH₂ etc.);

R^{A1} and R^{A2} are each independently selected from the groups

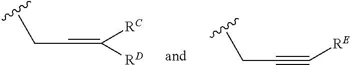

wherein R^{C} and R^{D} are each independently selected from hydrogen, deuterium, halogen and alkyl, and wherein R^{E} is hydrogen, deuterium, halogen or alkyl;
R^{B} is selected from R^{B1}, hydrogen and deuterium;
wherein R^{B1} is selected from phenyl, benzyl, heteroaryl, pyridyl, pyrimidyl and pyrazinyl optionally substituted independently with one or more substituents of R^{B2};
wherein each R^{B2} is independently selected from halogen, alkyl, substituted alkyl, deuterated alkyl, alkoxy, nitro, amino, methoxy, haloalkyl, polyhalogen alkyl, aminoalkyl, thioalkyl, alkoxy, haloalkoxy, and any atom or isotope permitted by valence (including any accompanying hydrogens by valence e.g. (non-limiting) OH, NH₂, SH, SiH₃, PH₂ etc.);
or R^{B} is a phenylalkyl of the formula:

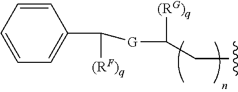

wherein R^{F} and R^{G} are hydrogen or alkyl, G is a carbon-carbon double bond or a carbon-carbon single bond, n is 0 or 1 and q is 0 or 1 provided that where q is 0, G is a carbon-carbon double bond and where q is 1, G is a carbon-carbon single bond,

or R^{B} is a diphenylalkyl of the formula

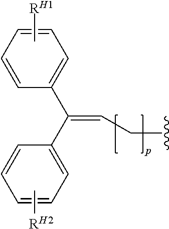

wherein R^{H1} and R^{H2} each independently represent 1-5 optional substituents on each ring, and wherein each R^{H1} and R^{H2}, when present, is independently selected at each point of use from hydrogen, deuterium, halogen, alkyl, substituted alkyl, deuterated alkyl, alkoxy, nitro, amino, methoxy, haloalkyl, polyhalogen alkyl, aminoalkyl, thioalkyl, alkoxy, haloalkoxy, and any atom or isotope permitted by valence (including any accompanying hydrogens by valence e.g. (non-limiting) OH, NH₂, SH, SiH₃, PH₂ etc.), and p is 0, 1, 2 or 3;

or R^{B} is the group

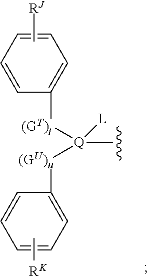

including

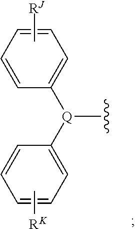

including

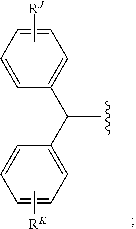

Wherein G^{T} and G^{U} are each independently selected from a single bond, O, S, NR^{V} or C(R^{V})₂, wherein each R^{V} is independently selected from hydrogen, deuterium, alkyl, substituted alkyl (non-limiting examples: CF₃, CCl₃), deuterated alkyl (non-limiting example: CD₃), aminoalkyl, thioalkyl, alkoxy, halogen (e.g. F), haloalkyl, haloalkoxy;

u and t are each independently selected from 0, 1, 2, 3 and 4;
Q is C, CH or N, R^{J} and R^{K} each independently represent 1-5 optional substituents on each ring, and wherein each R^{J} and each R^{K}, when present, is independently selected from deuterium, halogen, alkyl, substituted alkyl, deuterated alkyl, alkoxy, haloalkoxy, methoxy, nitro, amino, aminoalkyl, thioalkyl, haloalkyl, polyhalogen alkyl, and any atom or isotope permitted by valence (including any accompanying hydrogens by valence e.g. (non-limiting) OH, NH₂, SH, SiH₃, PH₂ etc.);
L is absent (when Q is N), alkyl, or substituted alkyl, or deuterated alkyl, or aminoalkyl, or thioalkyl, or alkoxy, or halogen, or haloalkyl, or haloalkoxy, or hydroxyalkyl, or any atom or isotope permitted by valence (including any accompanying hydrogens by valence e.g. (non-limiting) OH, NH₂, SH, SiH₃, PH₂ etc.).
In some embodiments, when one or both of R^{J} and R^{K} is alkoxy, this alkoxy group may be methoxy.
It is to be understood that in the compounds of general Formula (I), wherein R^{A1} and/or R^{A2} are alkenyl moieties having different substituents at the position R^{C} and R^{D}, that compound may exist in cis or trans isomeric forms both of which are considered to be within the scope of the present disclosure. All isotopic, including radionuclide, forms of Formula (I) are within the scope of the present disclosure.

Some Preferred Embodiments of Formula (I)

For Formula (I), the symbols R^{C} and R^{D}, as defined in subgroups R^{A1} and R^{A2}, may be hydrogen, halogen (suitably fluorine, chlorine or bromine), alkyl, suitably “lower alkyl” (herein now defined) having from 1 to 5 carbon atoms, such as methyl, ethyl, propyl, isopropyl, butyl, isobutyl, tert butyl, pentyl and the like, most preferably methyl;

and the moiety R^{E} may be hydrogen, or lower alkyl having from 1 to 5 carbon atoms such as methyl, ethyl, propyl, butyl, or pentyl, most suitably methyl.

The subgroup R^{B} may be hydrogen; phenyl; or substituted phenyl. The substituted phenyl group may include one or more of the preferred substituents in any of the available positions for substitution, however, mono substitution in the 4-position of the phenyl nucleus is especially preferred. Suitable substituents for the phenyl nucleus include halogen, preferably fluorine, chlorine or bromine; lower alkyl, lower alkoxy, and poly halogen lower alkyl (i.e. substituted alkyl) wherein the alkyl moiety contains from 1 to 5 carbon atoms, especially preferred however are methyl, methoxy, trifluoromethyl, nitro and amino Where the subgroup R^{B} represents substituted pyridyl, substituted pyrimidyl, or substituted pyrazinyl, the substituting group may be located on one or more of the available carbon atoms in the nucleus, and may be the same or different. Preferred among the substituting groups are lower alkyl or lower alkoxy having from 1 to 5 carbon atoms such as methyl, ethyl, butyl or penty; or methoxy, propoxy, butoxy or pentoxy. Where the moiety R^{B} represents substituted benzyl, the benzyl moiety may be substituted in one or more of the available positions on the phenyl nucleus thereof. Among the preferred substituents are halogen (suitably fluorine, chlorine or bromine), lower alkoxy having from 1 to 5 carbon atoms, especially preferred is methoxy and most preferred being di- and tri-methoxy; or alkylenedioxy suitably lower alkylenedioxy such as methylenedioxy, ethylenedioxy, propylenedioxy and the like, most suitably, the alkylenedioxy moiety is attached across the 3- and 4-positions of the phenyl nucleus, although the bridging of other carbon atoms in the phenyl nucleus is to be considered within the scope of the present disclosure.

The moieties R^{F} and R^{G} may be hydrogen, or lower alkyl of 1 to 5 carbon atoms, most preferred however being methyl.

The groups R^{H1} and R^{H2} may be independently hydrogen, or halogen suitably fluorine, chlorine or bromine.

Preferred embodiments of Formula (I) include wherein R^{C} and R^{D} are methyl, R^{E} is methyl and R^{B} is selected from chlorophenyl, methylphenyl, methoxyphenyl, trifluorophenyl, chlorophenyl, dimethoxybenzyl, trimethoxybenzyl, methylenedioxybenzyl and ethylenedioxybenzyl.

In some embodiments R^{B} is the group

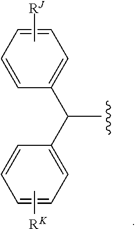

In some embodiments, R^{B} is the group

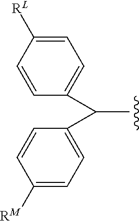

wherein R^{L} and R^{M} are each independently selected from halogen, alkyl, alkoxy, nitro, amino and polyhalogen alkyl.

Definitions Used to Specify Formula (I)

The term “alkyl” refers to straight or branched chain hydrocarbon groups having 1 to 21 carbon atoms, preferably 1 to 8 carbon atoms. Lower alkyl groups, that is, alkyl groups of 1 to 4 carbon atoms, are most preferred.

The term “substituted alkyl” refers to an alkyl group as defined above having one, two, three, or four substituents independently selected from the group consisting of PH₂, deuterium, halogen, trifluoromethyl, alkenyl, alkynyl, nitro, cyano, CH, keto (═O), ORₐ, SRₐ, NRₐR_{b}, NRₐSO₂, NRₐSO₂R_{c}, SO₂R_{c}, SO₂NRₐR_{b}, CO₂Rₐ, C(═O)Rₐ, C(═O)NRₐR_{b}, OC(═O)Rₐ, —OC(═O)NRₐR_{b}, NRₐC(═O)R_{b}, NRₐCO₂R_{b}, ═N—OH, ═N—O-alkyl, aryl, heteroaryl, heterocyclo and cycloalkyl, wherein Rₐ and R_{b} are independently selected from hydrogen, alkyl, alkenyl, cycloalkyl, heterocyclo, aryl, and heteroaryl, and R_{c} is selected from hydrogen, alkyl, cycloalkyl, heterocyclo aryl and heteroaryl. When a substituted alkyl includes an aryl, heterocyclo, heteroaryl, or cycloalkyl substituent, said ringed systems are as defined below and thus may in turn have zero to four independently selected substituents (preferably 0-2 substituents), also as defined below. When either Rₐ, R_{b} or R_{c} is an alkyl, said alkyl may optionally be substituted with 1-2 (selected independently) of PH₂, deuterium, halogen, trifluoromethyl, alkenyl, alkynyl, nitro, cyano, CH, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂,—OC(═O)NH(alkyl), NHC(═O)alkyl, and/or NHCO₂(alkyl).

“Alkyl” when used in conjunction with another group such as in arylalkyl refers to a substituted alkyl in which at least one of the substituents is the specifically named group. For example, the term arylalkyl includes benzyl, or any other straight or branched chain alkyl having at least one aryl group attached at any point of the alkyl chain. As a further example, the term carbamylalkyl includes the group —(CH₂)ₙ—NH—C(═O)alkyl, Wherein n is 1 to 12.

The term “alkenyl” refers to straight or branched chain hydrocarbon groups having 2 to 21 carbon atoms and at least one double bond. Alkenyl groups of 2 to 6 carbon atoms and having one double bond are most preferred.

The term “alkylene” refers to bivalent straight or branched chain hydrocarbon groups having 1 to 21 carbon atoms, preferably 1 to 8 carbon atoms, e.g., {—CH₂-}ₙ, Wherein n is 1 to 12, preferably 1-8. Lower alkylene groups, that is, alkylene groups of 1 to 4 carbon atoms, are most preferred. The terms “alkenylene” and “alkynylene” refer to bivalent radicals of alkenyl and alkynyl groups, respectively, as defined above.

When reference is made to a substituted alkylene, alkenylene, or alkynylene group, these groups are substituted with one to four substituents as defined above for alkyl groups. A substituted alkylene, alkenylene, or alkynylene may have a ringed substituent attached in a spiro fashion as in

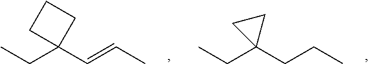

and so forth.

The term “alkoxy” refers to an alkyl or substituted alkyl group as defined above having one, two or three oxygen atoms (—O—) in the alkyl chain. For example, the term “alkoxy” includes the groups —O—C₁₋₁₂alkyl, —C₁₋₆alkylene-O—C₁₋₆alkyl, —C₁₋₄alkylene-O-phenyl, and so forth.

The term “thioalkyl” or “alkylthio” refers to an alkyl or substituted alkyl group as defined above having one or more sulphur (—S—) atoms in the alkyl chain. For example, the term “thioalkyl” or “alkylthio” includes the groups —CH₂SH, —(CH₂)ₙ—S—CH₂aryl, —(CH₂)ₙ—S-aryl, etc. etc.

The term “aminoalkyl” or “alkylamino” refers to an alkyl or substituted alkyl group as defined above having one or more nitrogen (—NR′—) atoms in the alkyl chain. For example, the term “aminoalkyl” includes the groups —NR′—C₁₋₁₂alkyl and —CH₂—NR′-aryl, etc. (where R′ is hydrogen, alkyl or substituted alkyl as defined above.) “Amino” refers to the group —NH₂.

When a subscript is used as in C₁₋₈alkyl, the subscript refers to the number of carbon atoms the group may contain. Zero when used in a subscript denotes a bond, e.g., C₀₋₄ alkyl refers to a bond or an alkyl of 1 to 4 carbon atoms. When used with alkoxy, thioalkyl or aminoalkyl, a subscript refers to the number of carbon atoms that the group may contain in addition to heteroatoms. Thus, for example, monovalent. C₁₋₂aminoalkyl includes the groups —CH₂—NH₂, —NH—CH₃, —(CH₂)₂—NH₂, —NH—CH₂—CH₃, —CH₂—NH₂—CH₃, and —N—(CH₃)₂. A lower aminoalkyl comprises an aminoalkyl having one to four carbon atoms.

The alkoxy, thioalkyl, or aminoalkyl groups may be monovalent or bivalent. By “monovalent” it is meant that the group has a valency (i.e., power to combine with another group), of one, and by “bivalent” it is meant that the group has a valency of two. For example, a monovalent alkoxy includes groups such as —O—C₁₋₁₂alkyl, —C₁₋₆alkylene-O—C₁₋₆alkyl, etc., whereas a bivalent alkoxy includes groups such as —O—C₁₋₁₂alkylene-, —C₁₋₆alkylene-O—C₁₋₆alkylene-, etc.

The term “acyl” refers to a carbonyl

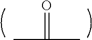

linked to an organic group i.e.

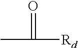

wherein R_{d} may be selected from alkyl, alkenyl, substituted alkyl, substituted alkenyl, aryl, heterocyclo, cycloalkyl, or heteroaryl, as defined herein.

The term “alkoxycarbonyl” refers to a group having a carboxy or ester group

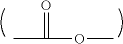

linked to an organic radical, i.e.,

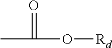

Wherein R_{d} is as defined above for acyl.

The term “cycloalkyl” refers to fully saturated and partially unsaturated hydrocarbon rings of 3 to 9, preferably 3 to 7 carbon atoms. The term “cycloalkyl” includes such rings having zero to four substituents (preferably 0-2 substituents), independently selected from the group consisting of OH, SH, PH₂, deuterium, halogen, alkyl, substituted alkyl (e.g., trifluoromethyl), alkenyl, substituted alkenyl, alkynyl, nitro, cyano, CH, keto, amino, alkoxy, hydroxy, methoxy, haloalkoxy, OCF₃, OR_{d}, SR_{d} NR_{d}Rₑ NR_{c}SO₂, NR_{c}SO₂Rₑ, C(═O)H, acyl, —CO₂H, alkoxycarbonyl, carbamyl, sulfonyl, sulfonamide, —OC(═O)R_{d}, ═N—OH, ═N—O-alkyl, aryl, heteroaryl, heterocyclo, a 4 to 7 membered carbocyclic ring, and a five or six membered ketal, e.g., 1,3-dioxolane or 1,3-dioxane, wherein R_{c}, R_{d} and Rₑ are defined as above. The term “cycloalkyl” also includes such rings having a phenyl ring fused thereto or having a carbon-carbon bridge of 3 to 4 carbon atoms. Additionally, when a cycloalkyl is substituted with a further ring, i.e., aryl, arylalkyl, heteroaryl, heteroarylalkyl, heterocyclo, heterocycloalkyl, cycloalkylalkyl, or a further cycloalkyl ring, such ring in turn may be substituted with one to two of C₀₋₄alkyl optionally substituted with one or more groups independently selected from OH, SH, PH₂, halogen, trifluoromethyl, alkenyl, alkynyl, nitro, cyano, CH, keto (═O), amino, alkoxy, hydroxy, methoxy, haloalkoxy, OCF₃, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF₃.

The term “aryl” refers to phenyl, biphenyl, 1-naphthyl, 2-naphthyl, and anthracenyl, with phenyl being preferred. The term “aryl” includes such rings having zero to four substituents (preferably 0-2 substituents), independently selected from the group consisting of deuterium, OH, SH, PH₂, halo, alkyl, substituted alkyl (e.g., trifluoromethyl), alkenyl, substituted alkenyl, alkynyl, nitro, cyano, amino, alkoxy, hydroxy, methoxy, haloalkoxy, OCF₃, CH, OR_{d}, SR_{d}, NR_{d}Rₑ, NR_{d}SO₂, NR_{d}SO₂R_{c}, C(═O)H, acyl, —CO₂H, alkoxycarbonyl, carbamyl, sulfonyl, sulfonamide, —OC(═O)R_{d}, heteroaryl, heterocyclo, cycloalkyl, phenyl, benzyl, napthyl, including phenylethyl, phenyloxy, and phenylthio, wherein R_{c}, R_{d} and Rₑ are defined as above. Additionally, two substituents attached to an aryl, particularly a phenyl group, may join to form a further ring such as a fused or spiro-ring, e.g., cyclopentyl or cyclohexyl or fused heterocyclo or heteroaryl. When an aryl is substituted with a further ring, such ring in turn may be substituted with one to two of C₀₋₄alkyl optionally substituted one or more groups independently selected from deuterium, SH, PH₂, halogen, trifluoromethyl, alkenyl, alkynyl, nitro, cyano, amino, alkoxy, hydroxy, methoxy, haloalkoxy, OCF₃, CH, keto (═O), OH, O(alkyl), phenyloxy, benzyloxy, SH, S(alkyl), NH₂, NH(alkyl), N(alkyl)2, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “heterocyclo” refers to substituted and unsubstituted non-aromatic 3 to 7 membered monocyclic groups, 7 to 11 membered bicyclic groups, and 10 to 15 membered tricyclic groups, in which at least one of the rings has at least one heteroatom selected from O, S and N. Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The fused rings completing bicyclic and tricyclic groups may contain only carbon atoms and may be saturated, partially saturated, or unsaturated. The nitrogen and sulfur atoms may optionally be oxidized and the nitrogen atoms may optionally be quaternized. The heterocyclo group may be attached at any available nitrogen or carbon atom. The heterocyclo ring may contain zero to four substituents (preferably 0-2 substituents), independently selected from the group consisting of deuterium, OH, SH, PH₂, halo, alkyl, substituted alkyl (e.g., trifluoromethyl), alkenyl, substituted alkenyl, alkynyl, nitro, cyano, amino, alkoxy, hydroxy, methoxy, haloalkoxy, OCF₃, CH, keto, OR_{d}, SR_{d}, NRₐRₑ, NR_{d}SO₂, NR_{d}SO₂Rₑ, SO₂R_{d}, C(═O)H, acyl, —CO₂H, alkoxycarbonyl, carbamyl, sulfonyl, sulfonamide, —OC(═O)R_{d}, ═N—OH, ═N—O-alkyl, aryl, heteroaryl, cycloalkyl, a five or six membered ketal, e.g., 1,3-dioxolane or 1,3-dioxane, or a monocyclic 4 to 7 membered non aromatic ring having one to four heteroatoms, wherein R_{c}, R_{d} and Rₑ are defined as above. The term “heterocyclo” also includes such rings having a phenyl ring fused thereto or having a carbon-carbon bridge of 3 to 4 carbon atoms. Additionally, when a heterocyclo is substituted with a further ring, i.e., aryl, arylalkyl, heteroaryl, heteroarylalkyl, cycloalkyl, cycloalkylalkyl, heterocycloalkyl, or a further heterocyclo ring, such ring in turn may be substituted with one to two of C₀₋₄alkyl optionally substituted with one or more groups independently selected from deuterium, SH, PH₂, halogen, trifluoromethyl, alkenyl, alkynyl, nitro, cyano, amino, alkoxy, hydroxy, methoxy, haloalkoxy, OCF₃, CH, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Exemplary monocyclic groups include azetidinyl, pyrrolidinyl, oxetanyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl.

The term “heteroaryl” refers to substituted and unsubstituted aromatic 5 to 7 membered monocyclic groups, 9 or 10 membered bicyclic groups, and 11 to 14 membered tricyclic groups which have at least one heteroatom selected from O, S and N in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero to four substituents (preferably 0-2 substituents), independently selected from the group consisting of deuterium, OH, SH, PH₂, halo, alkyl, substituted alkyl (e.g., trifluoromethyl), alkenyl, substituted alkenyl, alkynyl, nitro, cyano, amino, alkoxy, hydroxy, methoxy, haloalkoxy, OCF₃, CH, OR_{d}, SR_{d}, NR_{d}Rₑ, NR_{d}SO₂, NR_{d}SO₂R_{c}, SO₂R_{d}, C(═O)H, acyl, —CO₂H, alkoxycarbonyl, carbamyl, sulfonyl, sulfonamide, —OC(═O)R_{d}, heterocyclo, cycloalkyl, aryl, or a monocyclic 4 to 7 membered aromatic ring having one to four heteroatoms, including phenylethyl, phenyloxy, and phenylthio, wherein R_{c}, R_{d} and Rₑ are defined as above. Additionally, when a heteroaryl is substituted with a further ring, i.e., aryl, arylalkyl, heterocyclo, heterocycloalkyl, cycloalkyl, cycloalkylalkyl, heteroarylalkyl, or a further heteroaryl ring, such ring in turn may be substituted with one to two of C₀₋₄ alkyl optionally substituted with one or more groups independently selected from deuterium, PH₂, halogen, trifluoromethyl, alkenyl, alkynyl, nitro, cyano, amino, alkoxy, hydroxy, methoxy, haloalkoxy, OCF₃, CH, keto (═O), OH, O(alkyl), phenyloxy, benzyloxy, SH, S(alkyl), NH₂, NH(alkyl), N(alkyl)₂, NHSO₂, NHSO₂(alkyl)ₙ, SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Exemplary monocyclic heteroaryl groups include pyrrolyl, pyrazolyl, pyrazolinyl, imidazolyl, oxazolyl, isoxazolyl, thiazolyl

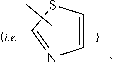

thiadiazolyl, isothiazolyl, furanyl, thienyl, oxadiazolyl, pyridyl, pyrazinyl, pyridinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

When the term “unsaturated” is used herein to refer to a ring or group, the ring or group may be fully unsaturated or partially unsaturated.

The phrase “optionally substituted” is intended to include substituted or unsubstituted possibilities. Accordingly, the phrase “each group of which may be optionally substituted” means that each group includes both substituted and unsubstituted groups. Where a list of substituents is specified for a group, unless specified otherwise, all substituent combinations permitted by valence are contemplated.

The term “substituted amino” refers to a group of the formula —NZ²Z³ wherein Z² is hydrogen, alkyl, cycloalkyl, aryl, arylalkyl, (cycloalkyl)alkyl, morpholinylalkyl, heterocyclo or (heterocyclo)alkyl and Z³ is hydrogen, alkyl, cycloalkyl, aryl, arylalkyl, haloalkyl, hydroxyalkyl, alkoxyalkyl, thioalkyl, (cycloalkyl)alkyl or hydroxyalkyl further substituted with a carboxylic ester and/or carboxylic acid, with the proviso that when Z² is hydrogen, then Z³ is other than hydrogen; or Z² and Z³ taken together with the nitrogen atom to which they are attached are 1-pyrrolidinyl, 1-piperidinyl, 1-azepinyl, 4-morpholinyl, 4-thiamorpholinyl, 1-piperazinyl, 4-alkyl-1-piperazinyl, 4-arylalkyl-1-piperazinyl, 4-diarylalkyl-1-piperazinyl; or 1-pyrrolidinyl, 1-piperidinyl, 1-azepinyl substituted with one or more groups independently selected from alkyl, alkoxy, alkylthio, halo, trifluoromethyl or hydroxy.

Hydroxy, hydroxyl and —OH are used interchangeably herein.

The term “heterocyclo” or “hetero” also includes such monocyclic and bicyclic rings wherein an available carbon atom is substituted with a (C₁-C₄)-alkyl, aryl, (C₁-C₄)-alkylthio, (C₁-C₄)-alkoxy, halo, nitro, keto, cyano, CH, hydroxy, azo, thiazo, amino, —NH—(C₁-C₄)-alkyl, —N((C₁-C₄)-alkyl)₂, —CF₃, (aminoester)alkyl, carboxylic acid, carboxylic ester, —OCHF₂ or (C₁-C₄)-alkoxy further substituted with a carboxylic acid or such monocyclic and bicyclic rings wherein two or three available carbons have substituents independently selected from methyl, methoxy, methylthio, halo, —CF₃, nitro, hydroxy, amino and —OCHF₂.

Herein, when “carboxyl” or “carboxylic acid” is used, this can mean —C(O)OH but can also refer to carboxylic ester or ester, which encompasses —OC(O)ORₑₛₜₑᵣ and —C(O)ORₑₛₜₑᵣ wherein Rₑₛₜₑᵣ is selected from hydrogen, deuterium, alkyl, alkenyl, alkoxy, thioalkyl, aminoalkyl, cycloalkyl, haloalkyl, haloalkoxy, aryl, heteroaryl or heterocyclo, each of which may be optionally substituted, as defined herein.

Use of “aryl” herein encompasses “aryloxy”, which refers to —O-aryl, wherein aryl is selected from prior definition of aryl specified herein. Use of “heteroaryl” herein encompasses “heteroaryloxy”, which refers to —O-heteroaryl, wherein heteroaryl is selected from prior definition of heteroaryl specified herein. Use of “heterocyclo” herein encompasses “hetereocyclooxy”, which refers to —O-heterocyclo, wherein heterocyclo is selected from prior definition of heterocyclo specified herein.

Alkylaryl groups are aryl groups substituted with one or more alkyl groups wherein the alkyl groups optionally carry additional substituents and the aryl groups are optionally substituted. Specific alkylaryl groups include alkyl-substituted phenyl groups such as methylphenyl.

At various places herein, substituents of compounds are disclosed in groups or in ranges. It is specifically intended that this disclosure include each and every individual sub-combination of the members of such groups and ranges. For example, the term “C₁₋₃alkyl” is intended to include C₁alkyl (methyl), C₂alkyl (ethyl), C₃alkyl.

### Synthesis of Compounds of Formula (I)

Synthesis routes for example embodiments of Formula (I) are in the following patent, which is herein incorporated in its entirety by reference: Regnier G, Canevari R, Laubie M, inventors; En Nom Collectif Science Union, Medicale Rech France, assignee. S-triazine compounds. U.S. Pat. No. 3,647,794. 1972, March 7.

Starting materials are commercially available (e.g. from one or more chemical suppliers listed on www.labnetwork.com {one inputs a chemical structure or name to find suppliers of it} and/or from a major chemical supplier known to those of the art e.g. Sigma-Aldrich, St. Louis, Mo., USA {which is a subsidiary of Merck KGaA} or similar) or can be readily prepared by one of ordinary skill in the art using known methods or derived by procedures analogous to those described in the literature

Almitrine dimesylate is commercially available.

### Isotopic Variants are Contemplated

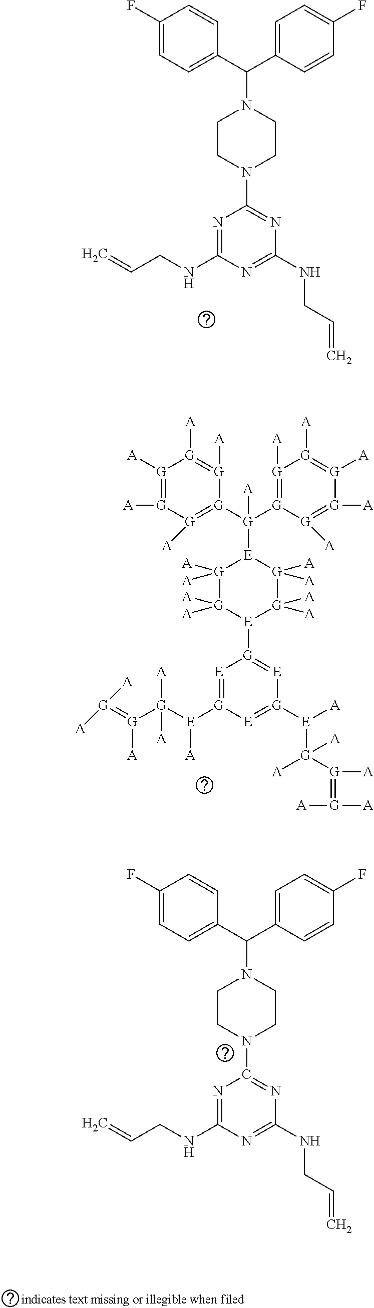

Difluorobenzhydrylpiperadine (DFBP) is the major almitrine metabolite formed in humans. Reducing DFBP formation increases the safety profile of almitrine. To render DFBP from almitrine, the bond between almitrine's nitrogen, at atom number 11, and carbon, at atom number 9, must be broken. An embodiment is almitrine isotopically enriched (greater than natural abundance, e.g. {non-limiting}>70%) for ¹⁵N at Atom Number 11, and/or isotopically enriched for ¹³C at Atom Number 9 (can be done, to illustrate and not limit, by substituting in cyanuric chloride-¹³C₃ {CAS: 286013-07-8; available from Sigma-Aldrich} for cyanuric chloride in almitrine synthesis, to give almitrine enriched at three carbon positions with ¹³C, including at Atom Number 9), which will make this bond stronger by the kinetic isotope effect (KIE), which will reduce the rate of DFBP formation (Atom Numbers as labelled by Marvinsketch software, made by ChemAxon).
Following reactions are illustrative, not restrictive: almitrine could be deuterated, upon its piperazine ring and/or other loci, by reactions described in [A], which deuterate sp3 carbons. And/or by reactions described in [B, C, D, E] which deuterate widely, upon aromatic and alkyl molecular components. And/or by reactions described in [F, G], which deuterate α- and β-carbons to phenyl groups. And/or by reactions described in [H], which deuterate α- and β-carbons to tertiary amines And/or by reactions described in [I, J, K, L], which deuterate α-carbons to tertiary amines And/or by reactions described in [M, N, I, J, K], which deuterate α-carbons to secondary amines Whichever option(s) is chosen, solvents, temperatures, pressures, and other reaction conditions can be selected by one of ordinary skill in the art. Deuteration can be modulated by modulating reaction time: greater deuterium incorporation by longer reaction time. One can do multiple cycles of one or more of these reactions until the desired level of deuterium incorporation occurs, monitored by ¹H and/or ²H NMR and/or mass spectrometry. All incorporated herein in their entirety by reference:
[A] Palmer W N, Chirik P J (2017) Cobalt-Catalyzed Stereoretentive Hydrogen Isotope 15 Exchange of C (sp3)-H Bonds. ACS Catalysis. 7(9): 5674-8. [B] Ito N et al. (2008) HD exchange reaction taking advantage of the synergistic effect of heterogeneous palladium and platinum mixed catalyst. Synthesis. 09:1467-78. [C] Maegawa T et al. (2009) Bimetallic Palladium-Platinum-on-Carbon-Catalyzed HD Exchange Reaction: Synergistic Effect on Multiple Deuterium Incorporation. Synthesis. (16):2674-8. [D] Derdau V, Atzrodt J (2006) CH/CD exchange reactions of aromatic compounds in D2O with NaBD4-activated catalysts. Synlett. (12): 1918-22. [E] Derdau V et al. (2009) Hydrogen-Deuterium Exchange Reactions of Aromatic Compounds and Heterocycles by NaBD4-Activated Rhodium, Platinum and Palladium Catalysts. Chemistry-α European Journal. 15(40):10397-404. [F] Sajiki H et al. (2004) Efficient C—H/C-D Exchange Reaction on the Alkyl Side Chain of Aromatic Compounds Using Heterogeneous Pd/C in D20. Organic letters. 6(9):1485-7. [G] Esaki H et al. (2007) Efficient H/D Exchange Reactions of Alkyl-Substituted Benzene Derivatives by Means of the Pd/C—H2-D2O System. Chemistry-A European Journal. 13(14):4052-63. [H] Neubert L et al. (2012) Ruthenium-catalyzed selective α,β-deuteration of bioactive amines. Journal of the American Chemical Society. 12: 134(29): 12239-44. [I] Bhatia S et al. (2016) Stereoretentive H/D Exchange via an Electroactivated Heterogeneous Catalyst at sp3 C—H Sites Bearing Amines or Alcohols. European Journal of Organic Chemistry. 24:4230-5. [J] Taglang C et al. (2015) Enantiospecific C—H Activation Using Ruthenium Nanocatalysts. Angewandte Chemie International Edition. 54(36): 10474-7. [K] Pieters G et al. (2014) Regioselective and stereospecific deuteration of bioactive aza compounds by the use of ruthenium nanoparticles. Angewandte Chemie International Edition. 53(1):230-4. [L] Loh Y Y et al. (2017) Photoredox-catalyzed deuteration and tritiation of pharmaceutical compounds. Science. eaap9674. [M] Takahashi M, Oshima K, Matsubara S (2005) Ruthenium catalyzed deuterium labelling of α-carbon in primary alcohol and primary/secondary amine in D2O. Chemistry letters. 34(2):192-3. [N] Chatterjee B et al. (2016) Selective α-Deuteration of Amines and Amino Acids Using D2O. Organic letters. 18(22):5892-5.

Treating Subjects with Cancer and a Coronavirus Infection

The present disclosure teaches that a therapeutically effective amount of at least one compound of Formula (I) (e.g. almitrine), or a pharmaceutically-acceptable salt thereof (e.g. almitrine dimesylate), can treat/ameliorate/prevent one or more symptoms (e.g. hypoxemia) of a coronavirus infection in a subject. Whilst my prior disclosure [11] teaches that almitrine (e.g. almitrine dimesylate) has anti-cancer activity: [11] Michael David Forrest. “Therapeutic Modulators of the Reverse Mode of ATP Synthase”. U.S. patent application Ser. No. 16/629,390. Published as US20200306253A1.

This patent application is herein incorporated in its entirety by reference (also, all its cited references are herein incorporated in their entirety by reference). All the teachings therein for almitrine (and/or almitrine dimesylate) administration for anti-cancer use are incorporated herein, and also applied to almitrine (and/or almitrine dimesylate) administration for treating/ameliorating/preventing one or more symptoms (e.g. hypoxemia) of a coronavirus infection in a subject that has cancer (or is suspected to have cancer, or that may have cancer), or that does not have cancer. In some embodiments, any feature/reference to Formula (VI) or Formula [X] therein (in [11]), applies to Formula (I) herein. In some embodiments, all the definitions therein (in [11]) apply herein also. For example, in some embodiments, its section titled “Definitions Used to Specify Formulas (I), (II), (III), (IV), (V), and (VI)”, starting from its paragraph [0671], on its page 67, gives definitions that are to be used herein, with Formula (I) herein (especially if any definition is missing in the present document). Of particular note, in some embodiments, incorporated into the present disclosure (wherein, in your reading of the following sections in [11], any reference to Formula (VI), and/or Formula [X], therein is to be recast as a reference to Formula (I) herein), is its sections: “Stereoisomers” (starting from its paragraph [0713]), “Salts, solvates, prodrugs” (starting from its paragraph [0717]), “Dosage” (starting from its paragraph [0736]), “Pharmaceutical composition” (starting from its paragraph [0740]), “Administration” (starting from its paragraph [0743]), “Co-administration” (starting from its paragraph [0749]). In [11], its paragraphs [0616] to [0670], especially its paragraphs [0659] to [0670], teach methods of almitrine (or almitrine dimesylate) administration for anti-cancer use, which, in some embodiments, are herein incorporated as they are in [11], but also, in parallel, incorporated in a form modified such that they apply to the administration of a subject with cancer and a coronavirus infection, and to a subject without cancer and just a coronavirus (e.g. SARS-CoV-2) infection. In some embodiments of this present disclosure, subject matter from [11] is read but with the caveat that wherever a subject with cancer is referred to (explicitly or implicitly), this is substituted with a subject with cancer and/or a coronavirus (e.g. SARS-CoV-2) infection instead (and the resulting teaching is componentry to the present disclosure). In some embodiments, any route of administration (e.g. oral or intravenous), and/or dose, and/or dosing regime/pattern, and/or salt, and/or pharmaceutical composition, of almitrine (and/or other compound(s) of Formula (I) herein) referred to at any point in (or one or more of its references therein), by the teaching of this present disclosure, can be applied to a subject with cancer and a coronavirus infection, or to a subject without cancer and just/only with a coronavirus (e.g. SARS-CoV-2) infection. Any information missing in this present document is not missing from this present disclosure because it can be found in [11] (and/or its cited references therein; especially, but not restrictively, refer to the aforementioned sections of [11; and/or in the present application's priority application[s]), with a modification to its content, such that the subject to be treated has cancer and a coronavirus infection, or does not have cancer but does have (or is suspected to have, or is at risk of) a coronavirus (e.g. SARS-CoV-2) infection.

Any patent application(s) that the present application claims priority from is herein incorporated in its entirety by reference. And all that it teaches.

Herein contemplated is a method comprising the administration of a therapeutically effective amount of at least one compound of Formula (I) (e.g. almitrine), and/or a pharmaceutically-acceptable salt, solvate, hydrate or prodrug thereof (e.g. almitrine dimesylate), and/or a pharmaceutical composition(s) comprising one or compounds of Formula (I), to a subject with (or suspected to have, or at risk of) cancer and with (or suspected to have, or at risk of), or only with, a coronavirus (e.g. SARS-CoV-2) infection (or other viral infection that can cause one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation, in an infected subject). Wherein this administration confers one or both of: (a) treatment/amelioration/prevention of the subject's cancer, and (b) the treatment/amelioration/prevention of one or more symptoms of the subject's coronavirus (e.g. SARS-CoV-2) infection e.g. at least one respiratory symptom (e.g. one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation). Without restriction, all the cancer types mentioned in [11] are contemplated herein. In some embodiments the subject's cancer is lung cancer (non-limiting e.g. Non-Small Cell Lung Cancer [NSCLC]), which can cause respiratory disorder(s) in and of itself. So, coronavirus infection is especially dangerous for lung cancer patients. They are a very high risk group for experiencing a bad clinical outcome from a coronavirus infection.

Co-Therapy with Nitric Oxide (NO)

Physiologically, hypoxia in part of a lung (e.g. perhaps because of lung damage and/or fluid in that part) causes vasoconstriction in this lung part (“Hypoxic Pulmonary Vasoconstriction”, HPV). So that more blood can flow instead to other lung parts that actually have appreciable O₂ to deliver to the blood. This vasoconstriction increases pulmonary tension. Pharmacologically, almitrine helps and increases this process. So increasing pO₂, and decreasing pCO₂, in the blood and tissues. Inherently increasing pulmonary tension. Breathable Nitric Oxide (NO) can be co-administered with almitrine. NO is a vasodilator. NO, when incorporated in the breathing mixture, only reaches the lung parts that O₂ reaches. So, it only vasodilates the lung parts that are well ventilated with O₂. So,
(1) Almitrine specifically vasoconstricts only hypoxic lung regions, shunting more blood to well ventilated lung regions.
(2) Breathed NO only reaches well ventilated lung regions and so specifically vasodilates only well ventilated lung regions.

Thence points, (1) and (2), additively increases PaO₂. Whilst point (1) increases pulmonary tension, point (2) decreases pulmonary tension. So, with almitrine and NO co-administration, there is partial/complete cancelling of their opposing pulmonary tension effects. With addition of their beneficial increase in PaO₂. Componentry to this disclosure is to co-administer almitrine (and/or other compound(s) of Formula (I); and/or a pharmaceutically acceptable salt, solvate, hydrate or prodrug thereof, e.g. almitrine dimesylate; non-limiting e.g. intravenous and/or oral) with breathed NO to a subject, wherein this subject has cancer and/or a coronavirus infection, or only a coronavirus infection. Where the breathed NO permits a higher almitrine dose(s) (e.g. conferring greater anti-cancer activity) to be administered to the subject. Because the breathed NO counteracts an almitrine conferred increase in pulmonary tension. Wherein a higher NO dose can permit a higher almitrine dose.

Also componentry to the present disclosure is to co-administer almitrine (and/or other compound(s) of Formula (I); and/or a pharmaceutically acceptable salt, solvate, hydrate or prodrug thereof, e.g. almitrine dimesylate; non-limiting e.g. intravenous and/or oral) and breathed NO (optionally along with artificial/assisted/mechanical ventilation {optionally with intubation} and/or hyperbaric/supplemental oxygen (oxygen therapy) and/or postural positioning [e.g. prone, supine] and/or Extracorporeal Membrane Oxygenation [ECMO]) to a subject infected with at least one coronavirus, optionally SARS-CoV-2, or to a subject with cancer (e.g. lung cancer {e.g. NSCLC}) and/or a coronavirus (e.g. SARS-CoV-2) infection. Optionally to treat/ameliorate/prevent/combat a coronavirus driven/associated/correlated respiratory disorder(s): optionally one or more of breathing disorder/difficulty, shortness of breath/breathlessness, dyspnoea, low PaO₂, hypoxemia, refractory hypoxemia, hypoxia, hypoxemic hypoxia, anoxemia, hypoxemia with minimal to no dyspnea [“silent hypoxemia”], hypercapnia, pneumonia, pneumonitis, Acute Respiratory Distress Syndrome [ARDS], Severe Acute Respiratory Distress Syndrome [SARDS], ARDS or SARDS type syndrome, alveolar hypoventilation. Particularly hypoxemia.

At any point that almitrine (and/or almitrine dimesylate) administration is referred to in this disclosure, in further embodiments of this disclosure, NO (non-limiting e.g. at 10 ppm) is administered also.

Stereoisomers

Refer to section titled “Stereoisomers” of [11] (US20200306253A1), which starts from its paragraph [0713], herein incorporated by reference, substituting its “Formula [X]” for “Formula (I)”, such that it refers to Formula (I) herein, in the present document.

Salts & Solvates

Refer to section titled “Salts, solvates, prodrugs” of [11] (US20200306253A1), which starts from its paragraph [0717], herein incorporated by reference, substituting its “Formula [X]” for “Formula (I)”, such that it refers to Formula (I) herein, in the present document.

Dosage

Refer to section titled “Dosage” of [11] (US20200306253A1), which starts from its paragraph [0736], herein incorporated by reference, substituting its “Formula [X]” for “Formula (I)”, such that it refers to Formula (I) herein, in the present document.

Pharmaceutical Composition

Refer to section titled “Pharmaceutical composition” of [11] (US20200306253A1), which starts from its paragraph [0740], herein incorporated by reference, substituting its “Formula [X]” for “Formula (I)”, such that it refers to Formula (I) herein, in the present document.

Administration

Refer to section titled “Administration” of [11] (US20200306253A1), which starts from its paragraph [0743], herein incorporated by reference, substituting its “Formula [X]” for “Formula (I)”, such that it refers to Formula (I) herein, in the present document.
For non-limiting example, almitrine administered to the subject via their respiratory system (e.g. via their inhaling, or via the subject breathing a breathing gas of which almitrine is a component [optionally Nitric Oxide, e.g. at 10 ppm, is componentry also]).

Co-Administration

Refer to section titled “Co-administration” of [1] (US20200306253A1), which starts from its paragraph [0749], herein incorporated by reference, substituting its “Formula [X]” for “Formula (I)”, such that it refers to Formula (I) herein, in the present document.

Some Further Almitrine Forms for Use

A disclosure embodiment(s) is a pharmaceutical composition comprising a therapeutically effective amount of at least one compound of Formula (I) (e.g. almitrine, and/or a pharmaceutically-acceptable salt, solvate, hydrate or prodrug thereof, e.g. almitrine dimesylate) and a fatty acid(s) and/or cyclodextrin(s). Such complexes are taught in my earlier, published Australian (AU) patent application (2019208238, “Therapeutic Modifiers of the Reverse Mode of ATP Synthase”, Inventor/Applicant: Michael David Forrest), herein incorporated in its entirety by reference, wherein all that it teaches about almitrine (and other compounds of Formula (I) herein, which corresponds to Formula (VI) therein) for anti-cancer use (non-limiting e.g. salts, solvates, prodrugs, liposomes, nanoparticles etc. thereof, dosages, pharmaceutical compositions, routes of administration, crystalline forms, micronized forms, controlled release forms, fast melt formulations, kits etc.) is incorporated herein for treating subjects with cancer and a coronavirus infection, and subjects without cancer and just with a coronavirus infection (e.g. SARS-CoV-2). These teachings also particularly apply to a pharmaceutical composition comprising almitrine and one or both of dexamethasone and remdesivir.

Some Definitions, and a Comment on Scope

If there is any contradiction of definition(s) in this disclosure, all the given definitions are valid but for different embodiments of the disclosure. Where a term is provided in the singular, the inventor also contemplates the plural of that term. Where a term is provided in the plural, the inventor also contemplates the singular of that term. A phrase in the form “A/B” or in the form “A and/or B” means (A), (B), or (A and B). Herein, wherever “and” is used, in an alternative embodiment(s), “or” is used/substituted in its place. And wherever “or” is used, in an alternative embodiment(s) “and” is used/substituted in its place. As used herein with reference to the utilities described, the terms “treating” or “treatment” encompass both responsive and prophylaxis/preventative measures designed to inhibit/eradicate/prevent, reduce risk of and/or delay the onset/cause of the disease or disorder (or one or more of its symptoms), or to cure/eradicate, alleviate, abrogate, palliate, reverse, prevent, ameliorate, lessen, reduce, modulate, stabalize, delay, suppress, manage, reduce predisposition to, reduce risk of, prevent, reduce reoccurrence of, lengthen time to remission of, or slow progression/spread of the disease or disorder and/or one or more of its symptoms and/or increase quality/length of life and/or improve subject outcome/wellness. The terms “subject” and “patient” refer to organisms to be treated by the compounds/methods of the present disclosure and can refer to a human or animal. The terms “subject′ and “patient” are used interchangeably herein, in reference, for example, to a mammalian subject, such as a human patient. The term “subject” refers to an animal, including, but not limited to, a primate (e.g. human, monkey, chimpanzee, gorilla, and the like), a rodent (e.g. rat, mouse, gerbil, hamster, ferret, and the like), a lagomorph, a swine (e.g. pig, miniature pig), an equine, a canine, a feline, and the like, a companion/exotic/farm/laboratory animal. As used herein, the term “therapeutically effective amount” or “effective amount” refers to the amount of a compound (e.g. a compound of the present disclosure) sufficient to effect a therapeutically/aesthetically beneficial/desired result including, for example, mitigating/alleviating to some extent (reducing frequency/duration/severity, and/or prevent development of) or eliminating one or more symptoms of the disease/disorder/condition/sub-optimum, or treating at least one physiological defect or pathology or etiology that causes or contributes to the disease/disorder/condition/sub-optimum being treated. In some embodiments, where the word “subject” is used in a sentence of this disclosure, it is substituted with “subject in need of treatment” or “subject in need thereof” or “subject in need/want thereof”. Three different claim types: method of medical treatment, Swiss-type and Product by process (purpose-limited-product format, EPC 2000); in this disclosure, when a claim or statement is given in one of these forms it also incorporates by reference the same subject matter in both the other claim forms.

This disclosure encompasses all combinations of aspects of the disclosure noted herein. It is understood that any and all embodiments of the present disclosure may be taken in conjunction with any other embodiment or embodiments to describe additional embodiments. It is also to be understood that each individual element of the embodiments is its own independent embodiment. Furthermore, any element of an embodiment is meant to be combined with any and all other elements from any embodiment to describe an additional embodiment. Feature(s) described in connection with one embodiment of the disclosure may be used in conjunction with another embodiment(s), even if not explicitly stated. Any/all of the features described herein (including any accompanying claims, abstract and drawings), and/or all/some of the steps of any method or process so disclosed, may be combined with any of the above aspects in any combination.

1. Almitrine, and/or a pharmaceutically-acceptable salt, solvate, hydrate or prodrug thereof, e.g. almitrine dimesylate, for use in a method of treating/ameliorating/preventing a coronavirus infection in a subject.

2. Almitrine, and/or a pharmaceutically-acceptable salt, solvate, hydrate or prodrug thereof, e.g. almitrine dimesylate, for use in a method of treating/ameliorating/preventing at least one respiratory symptom of a coronavirus infection in a subject.

3. A compound for use according to claim 2 wherein the respiratory symptom is breathing difficulty.

4. A compound for use according to claim 2 wherein the respiratory symptom is Acute Respiratory Distress Syndrome (ARDS) or Severe Acute Respiratory Distress Syndrome (SARDS).

5. Almitrine, and/or a pharmaceutically-acceptable salt, solvate, hydrate or prodrug thereof, e.g. almitrine dimesylate, for use in a method of treating/ameliorating/preventing the hypoxemia (low PaO₂, low blood PO₂) of a coronavirus infection in a subject.

6. A compound for use according to one or more of claims 1-5 wherein the subject is human.

7. A compound for use according to one or more of claims 1-6 wherein the coronavirus is SARS-CoV-2.

8. A compound for use according to one or more of claims 1-6 wherein almitrine dimesylate is administered to the subject orally.

9. A compound for use according to one or more of claims 1-6 wherein almitrine dimesylate is administered to the subject intravenously.

10. A compound for use according to one or more of claims 1-9 wherein Nitric Oxide (NO) is co-administered, via the subject's breathing gas, optionally at 10 parts per million (ppm).

11. A compound for use according to one or more of claims 1-10 wherein the subject receives supplemental O₂ (oxygen therapy) whilst they have an effective amount of almitrine in their body.

12. A compound for use according to one or more of claims 1-11 wherein the subject is mechanically/artificially ventilated (optionally invasively) whilst they have an effective amount of almitrine in their body.

13. A compound for use according to claim 12 wherein its administration delays, or removes, the need for mechanical ventilation [invasive or non-invasive], or shortens the duration of mechanical ventilation [invasive or non-invasive] required;
wherein its administration reduces the probability that the subject will require mechanical ventilation [invasive or non-invasive].

14. A compound for use according to claim 12 wherein its administration is used to wean/expedite the subject off mechanical ventilation [invasive or non-invasive].

15. A compound for use according to one or more of claims 1-11 wherein the subject undergoes Extracorporeal Membrane Oxygenation (ECMO) whilst they have an effective amount of almitrine in their body.

16. A compound for use according to claim 15 wherein its administration delays, or removes, the need for Extracorporeal Membrane Oxygenation (ECMO), or shortens the duration of ECMO required;
wherein its administration reduces the probability that the subject will require ECMO.

17. A compound for use according to claim 15 wherein its administration is used to wean/expedite the subject off ECMO.

18. A compound for use according to one or more of claims 1-17 wherein the subject has cancer.

19. A compound for use according to claim 1-18 wherein the subject has lung cancer, optionally Non-Small Cell Lung Cancer (NSCLC).

20. A compound for use according to one or more of claims 18-19 wherein the administered compound treats/ameliorates the subject's cancer(s).

21. A compound for use according to one or more of claims 1-20 wherein the administered compound is an isotopologue, optionally isotopically enriched for deuterium at one or more positions.

22. A compound for use according to one or more of claims 1-21 wherein both almitrine and almitrine dimesylate (and/or isotopic variant[s] thereof) are administered in combination to the subject.

23. A compound for use according to one or more of claims 1-22 wherein the administered compound delays the need for the subject to enter hospital, and/or expedites their discharge from hospital, or removes/mitigates the need for the subject to go to hospital.

24. A compound for use according to claim 23 wherein the subject with coronavirus spending less or no time in hospital reduces the spread of coronavirus within the hospital, and thence the broader population (reduces subject's infectivity, reduces the number of other individuals [e.g. people] that the subject infects with coronavirus; when done for a population of coronavirus infected subjects it reduces the Reproduction (R) number of coronavirus transmission, which can slow the growth rate of a coronavirus outbreak/epidemic/pandemic).

25. A compound for use according to one or more of claims 1-24 wherein the subject self-administers the compound in the absence of a medical practitioner.

26. A compound for use according to one or more of claims 1-25 wherein the subject is also administered with one or more respiratory stimulation drugs (e.g. doxapram) and/or one or more anti-viral drugs (e.g. one or more of remdesivir, lopinavir, ritonavir etc., wherein remdesivir is particularly preferred).

27. A compound for use according to one or more of claims 1-26 wherein the subject's chance of survival is increased; wherein their probability of dying from their coronavirus infection is decreased.

28. A compound for use according to one or more of claims 1-27 wherein the subject's time to recovery is decreased (e.g. they are discharged from hospital earlier)
and/or, if they require respiratory support, the duration that they require respiratory support is decreased.

29. A compound for use according to one or more of claims 1-28 wherein almitrine or almitrine dimesylate is administered to a subject with a coronavirus infection, optionally who is requiring respiratory support (e.g. mechanical ventilation [invasive or non-invasive] e.g. the invasive sub-type thereof), wherein the subject is also administered with dexamethasone and/or remdesivir;
preferably such that the subject has a therapeutically effective amount of almitrine and [dexamethasone and/or remdesivir] in their body at the same time;
optionally wherein almitrine and [dexamethasone and/or remdesivir] are administered to the subject in the same pharmaceutical composition;
preferably wherein this co-administration reduces the duration that the subject requires respiratory support and/or improves the subject's recovery time (e.g. quickens hospital discharge) and/or improves the subject's chance of survival and/or improves the subject's clinical outcome, preferably better than any of these drugs could do alone.
